(19) United States
(12) Patent Application Publication
(10) Pub. No.: US 2025/0320208 A1
(43) Pub. Date: Oct. 16, 2025

(54) Intermediates of Sonrotoclax and the Method of Preparing the Same

(71) Applicant: BeOne Medicines I GmbH, Basel (CH)

(72) Inventors: Kai Peng, Suzhou (CN); Zhengfeng Li, Suzhou (CN); Tao Zhang, Suzhou (CN); Juntao Liu, Suzhou (CN); Qun Jiang, Suzhou (CN)

(73) Assignee: BeOne Medicines I GmbH, Basel (CH)

(21) Appl. No.: 19/250,664

(22) Filed: Jun. 26, 2025

Related U.S. Application Data
(63) Continuation of application No. PCT/CN2023/141985, filed on Dec. 26, 2023.

(30) Foreign Application Priority Data
Dec. 27, 2022 (WO) PCT/CN2022/142314

(51) Int. Cl. C07D 471/04; C07D 519/00

(52) U.S. Cl. CPC C07D 471/04; C07D 519/00

(57) ABSTRACT

Disclosed herein are compounds of Formula (I) having protective group of carbonyl used as intermediates for preparing 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Sonrotoclax), and processes for preparing compounds of Formula (I), including methyl 2-((1H-pyrrolo[2,3-b]pyridin-5-yl)oxy)-4-(1,5-dioxa-11-azadispiro[5.1.5⁸.1⁶]tetradecan-11-yl)benzoate.

### CROSS-REFERENCE TO RELATED APPLICATIONS

This application is a continuation of International Application No. PCT/CN2023/141985, filed on Dec. 26, 2023, which claims priority to International Application No. PCT/CN2022/142314, filed on Dec. 27, 2022, the disclosures of each of which are hereby incorporated by reference in their entireties.

### FIELD

Disclosed herein are compounds of Formula (I) having a carbonyl protective group used as intermediates for preparing 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Sonrotoclax), and processes for preparing compounds of Formula (I), including methyl 2-((1H-pyrrolo[2,3-b]pyridin-5-yl)oxy)-4-(1,5-dioxa-11-azadispiro[5.1.5⁸.1⁶]tetradecan-11-yl)benzoate.

### BACKGROUND

International publication WO2019/210828 disclosed a series of Bcl-2 inhibitors, in particular, 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (hereinafter Sonrotoclax), which selectively inhibit Bcl-2 proteins for the treatment of dysregulated apoptotic diseases such as cancers, autoimmune diseases and pro-thrombotic conditions.

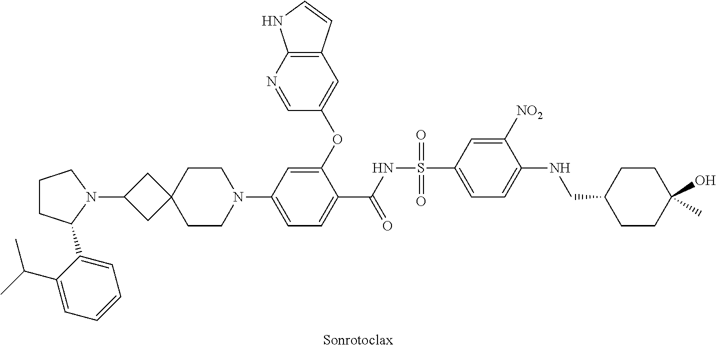 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

However, the process of preparing Sonrotoclax also needs to be optimized. The current yield of the reaction is relatively low and the current intermediates are not stable enough for the industrial process. Therefore, there is a need for novel reaction intermediates and processes for preparing Sonrotoclax.

### SUMMARY

Provided herein are compound of Formula (I),

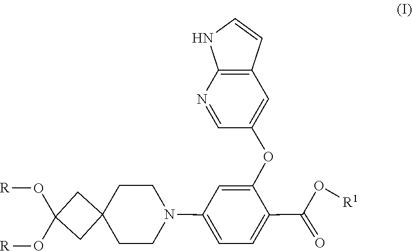

and salts, stereoisomers, and derivatives thereof. Further provided herein are methods of making the compound of Formula (I). Further provided herein are methods of making Sonrotoclax from the compound of Formula (I).

### DETAILED DESCRIPTION

The following terms have the indicated meanings throughout the specification:

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

An “alkynyl” group refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₈ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1-and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers.

Throughout this specification and the Aspects which follow, the term “Cₙ₋ₘ” indicates a range which includes the endpoints, wherein n and m are integers and indicate the number of carbons. Examples include C₁₋₈, and C₁₋₆.

In the claims which follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

Provided herein are intermediates which can greatly increase the yield and purity of Sonrotoclax; and are also stable enough to provide a robust process for industry. The inventors have found that Sonrotoclax can be obtained in sold state with a purity of more than 90% and yield of more than 70%.

In one embodiment, disclosed herein is a compound of Formula (I). The embodiment comprises the following aspects:

Aspect 1. A compound of Formula (I):

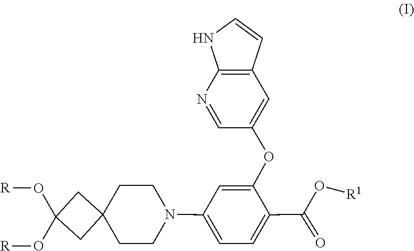

or a salt thereof, or a stereoisomer thereof,
wherein
at each of its occurrences, R is each independently C₂₋₆alkyl or C₃₋₆cycloalkyl, each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
R¹ is C₁₋₆alkyl, or C₃₋₆cycloalkyl, wherein each of said C₁₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl.

In particular, methyl 2-((1H-pyrrolo[2,3-b]pyridin-5-yl)oxy)-4-(1,5-dioxa-11-azadispiro[5.1.5⁸.1⁶]tetradecan-11-yl)benzoate is a significant intermediate for the synthesis of Sonrotoclax and said intermediate can be obtained by the SNAr reaction between 1,5-dioxa-11-azadispiro[5.1.5⁸.1⁶]tetradecane hydrochloride and methyl 2-((1H-pyrrolo[2,3-b]pyridin-5-yl)oxy)-4-fluorobenzoate aided with a base. The synthesis and process of methyl 2-((1H-pyrrolo[2,3-b]pyridin-5-yl)oxy)-4-(1,5-dioxa-11-azadispiro[5.1.5⁸.1⁶]tetradecan-11-yl)benzoate (Compound 1) is highly desirable, because Compound 1 as an intermediate with a high purity and high yield can meet the requirements for the manufacture of the final product Sonrotoclax.

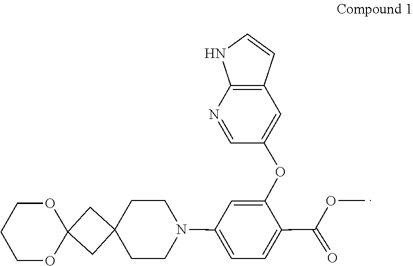 [SMILES: COC(=O)c1ccc(N2CCC3(CC2)CC2(C3)OCCCO2)cc1Oc1cnc2[nH]ccc2c1]

Aspect 2. The compound of Aspect 1, wherein
R is each independently ethyl, propyl, butyl, pentyl, hexyl, cyclopropyl, cyclobutyl, cyclopentyl, or cyclohexyl; said ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl; or
two R together with the two oxygen atoms to which each is attached, form a 3- to 12-membered ring, said ring is optionally substituted with at least one substituent said methyl, ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl;
and R¹ is each independently ethyl, propyl, butyl, pentyl, hexyl, cyclopropyl, cyclobutyl, cyclopentyl, or cyclohexyl; said ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl.

Aspect 3. The compound of Aspect 1, wherein
R is each independently methyl, ethyl, 1-propyl, or 2-propyl; or
two R together with the two oxygen atoms to which each is attached form a 3-, 5-, 6-, 7-, or 8-membered ring;
R¹ is methyl, ethyl, 1-propyl, or 2-propyl.

Aspect 4. The compound of Aspect 2, wherein two R together with the two oxygen atoms to which each is attached form a ring which contains 3 carbon atoms or more; and
R¹ is methyl.

Aspect 5. The compound of Aspect 1, having the structure below:

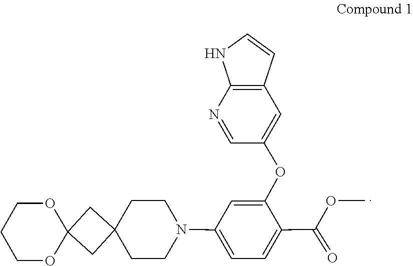 [SMILES: COC(=O)c1ccc(N2CCC3(CC2)CC2(C3)OCCCO2)cc1Oc1cnc2[nH]ccc2c1]

Aspect 6. A method for preparing the compound of Formula (I) according to any one of Aspects 1-5, said method comprising:

The compound of Formula (II) and the compound of Formula (III) are reacted in the presence of base and solvent to form the compound of Formula (I);

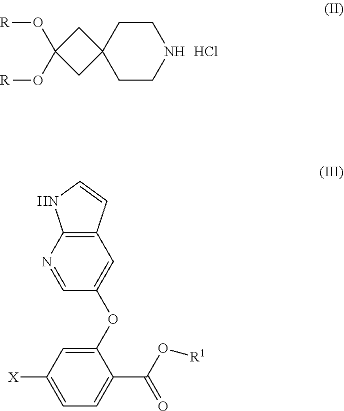

wherein
X is halogen; preferably F, Cl, Br, or I; preferably, F.

To optimize the synthesis of Sonrotoclax and to yield a more stable intermediate of Formula (II), pharmaceutically acceptable alcohols including methanol, ethanol, isopropanol, glycol and 1,3-propandiol have been used in ketone protection of 1-azaspiro[3.5]nonan-2-one. The inventors found that, among those alcohols, only 1,3-propandiol produced 1,5-dioxa-11-azadispiro[5.1.5⁸.1⁶]tetradecane hydrochloride (compound 2) in solid state with a good physical form and a high purity, of even 99.9%. In addition, compound 2 in solid state is also found to be highly stable with no increase of impurities when stored under 2-8° C. for 3.5 months or longer.

Aspect 7. The method of Aspect 6, wherein
the base is selected from the group consisting of: 1,8-Diazabicyclo[5.4.0]undec-7-ene (DBU), 1,1,3,3-Tetramethylguanidine (TMG), 1,5-Diazabicyclo[4.3.0]non-5-ene (DBN), 1,4-Diazabicyclo[2.2.2]octane (DABCO), me-piperidine and N,N-Diisopropylethylamine (DIPEA), or the combination thereof;
preferably, 1,8-Diazabicyclo[5.4.0]undec-7-ene (DBU), 1,1,3,3-Tetramethylguanidine (TMG), 1,5-Diazabicyclo[4.3.0]non-5-ene (DBN), or the combination thereof,
more preferably, 1,8-Diazabicyclo[5.4.0]undec-7-ene (DBU).

Aspect 8. The method of any one of Aspects 6-7, wherein
the molar ratio of the base used in the reaction to the compound of Formula (II) is 0.9-1.3;
preferably, the molar ratio of the base used in the reaction to the compound of Formula (II) is about 1.

Aspect 9. The method of any one of Aspects 6-8, wherein
the solvent is selected from the group consisting of: N-methylpyrrolidone(NMP), dimethyl sulfoxide(DMSO), 2-methyltetrahydrofuran (2-MeTHF), 4-methyl-2-pentanone (MIBK) or toluene, or the combination thereof,
preferably, N-methylpyrrolidone(NMP).

In a further embodiment, NMP gave better conversion and low impurity.

Aspect 10. The method of any one of Aspects 6-9, wherein
the weight ratio of the solvent used in the reaction to the summation of the compound of Formula (II) and the compound of Formula (III) is about 4-6;
preferably, about 5.

Aspect 11. The method of any one of Aspects 6-10, wherein the amount of the compound of Formula (II) used in the reaction ranges from about 1.3 molar equivalents to about 1.8 molar equivalents relative to the amount of the compound of Formula (III);
preferably, about 1.45 molar equivalents to about 1.65 molar equivalents.

Aspect 12. The method of any one of Aspects 6-11, wherein the reaction temperature range is from about 40° C. to about 100° C.;
preferably, about 65° C. to about 80° C.;
more preferably, about 75° C.

Aspect 13. The method of any one of Aspects 6-12, said method comprising
Step 1): the compound of Formula (II) and the compound of Formula (III) are reacted in the presence of a base and a solvent;
Step 2): after the reaction of step 1) is completed, water, AcOH and the crystal seed of formula (I) are added to the reaction system; after being filtered, the crude product containing the compound of Formula (I) is obtained;
Step 3): the crude product containing the compound of Formula (I) is slurried with a mixture of solvents, filtered and dried to obtain the compound of Formula (I).

In an embodiment, the crystal seed of formula (I) was prepared by the method below comprising:
Step 1-1): the compound of Formula (II) and the compound of Formula (III) are reacted in the presence of base and solvent;
Step 2-1): after the reaction of step 1) is completed, water, and AcOH are added to the reaction system; after being filtered, the crude product containing the compound of Formula (I) is obtained;
Step 3-1): the crude product containing the compound of Formula (I) is slurried with a mixture of solvents, filtered and dried to obtain the crystal seed of formula (I).

Aspects 14. The method of Aspect 13, wherein the mixture of solvents comprises 2-MeTHF and N-heptane.

Aspects 15. The method of Aspects 14, wherein 2-MeTHF: n-heptane(v:v)=1-4:1.

Aspects 16. Provided herein is a method of making Sonrotoclax. In one embodiment, the method comprises reacting the compound of Formula (I) with an acid or a base to provide a compound of Formula (SI),

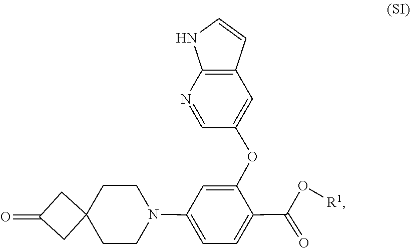

In one embodiment, the acid is HCl, or the base is NaOH.

Aspects 17. Provided herein is a method of making Sonrotoclax. In one embodiment, the method comprises reacting the compound of Formula (SI) with (S)-2-(2-isopropylphenyl)pyrrolidine, or a salt thereof, to provide a compound of Formula (SII)

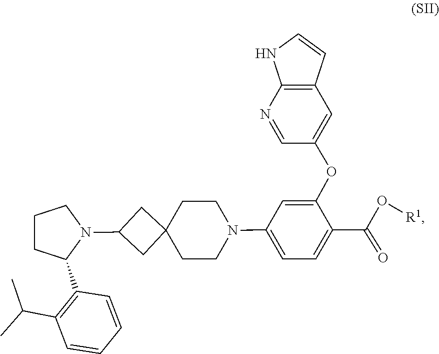

or a salt thereof.

In one embodiment, the reaction happens in the presence of NaBH(OAc)₃.

In one embodiment, the reaction happens at less than about 30° C.

Aspects 18. Provided herein is a method of making Sonrotoclax. In one embodiment, the method comprises reacting the compound of Formula (SII)

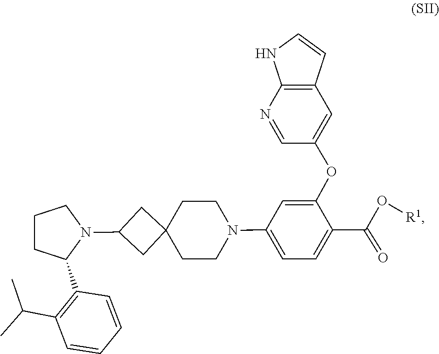

or a salt thereof, with 4-((((1r,4r)-4-hydroxy-4-methylcyclohexyl)meth y)amino)-3-nitrobenzenesulfonamide, to provide Sonrotoclax,

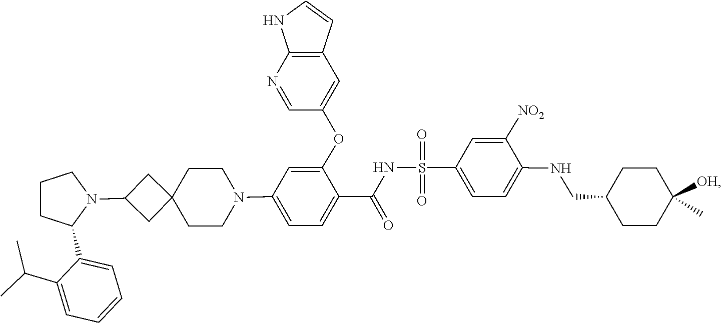 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof.

In one embodiment, the reaction happens in the presence of 1-ethyl-3-(3-dimethylaminopropyl)carbodiimide (EDCI) and 4-dimethylaminopyridine (DMAP).

Aspects 19. Provided herein is a method of making Sonrotoclax. In one embodiment, the method comprises reacting the compound of Formula (SII) with an acid or a base, to provide (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid,

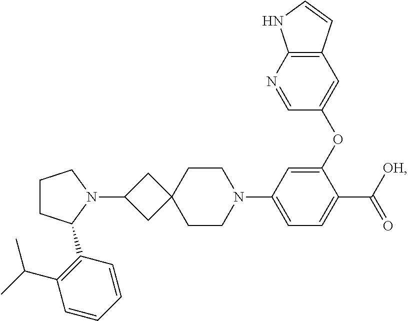 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof.

In one embodiment, the acid is HCl, or the base is NaOH.

Aspects 20. Provided herein is a method of making Sonrotoclax. In one embodiment, the method comprises (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid,

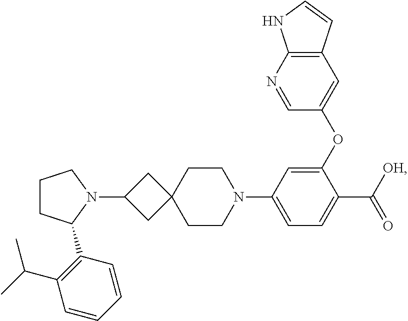 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof, with 4-((((1r,4r)-4-hydroxy-4-methylcyclohexyl)methyl)amino)-3-nitrobenzenesulfonamide, to provide Sonrotoclax,

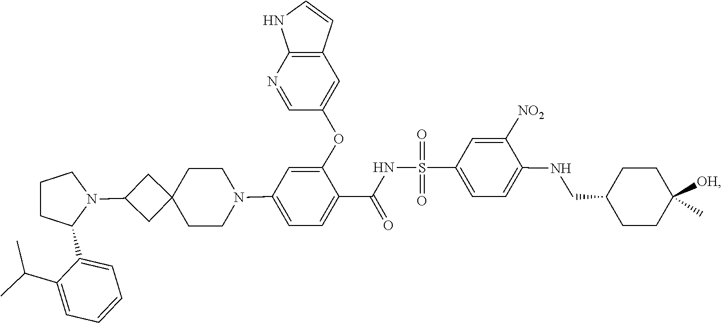 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof.

In one embodiment, the reaction happens in the presence of EDCI and DMAP.

In one embodiment, provided here is a method for preparing a pharmaceutical composition comprising Sonrotoclax, comprising mixing Sonrotoclax with a pharmaceutically acceptable excipient, wherein Sonrotoclax is prepared according to the method provided here.

Unless specifically defined elsewhere in this document, all other technical and scientific terms used herein have the meaning commonly understood by one of ordinary skill in the art.

### Numbered Embodiments

Embodiment 1. A compound of Formula (I),

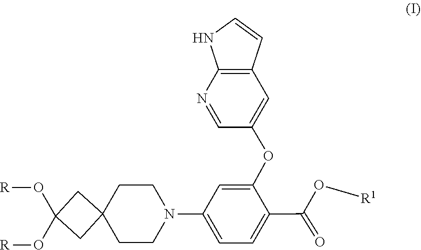

or a salt thereof, or a stereoisomer thereof,
wherein
at each of occurrences, R is each independently C₂₋₆alkyl or C₃₋₆cycloalkyl, each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
R¹ is C₁₋₆alkyl, or C₃₋₆cycloalkyl, wherein each of said C₁₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl.

Embodiment 2. The compound of embodiment 1, wherein:
R is each independently ethyl, propyl, butyl, pentyl, hexyl, cyclopropyl, cyclobutyl, cyclopentyl, or cyclohexyl; said ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl; or
two R together with the two oxygen atoms to which each is attached, form a 3- to 12-membered ring, said ring is optionally substituted with at least one substituent said methyl, ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl;
R¹ is each independently methyl, ethyl, propyl, butyl, pentyl, hexyl, cyclopropyl, cyclobutyl, cyclopentyl, or cyclohexyl; said ethyl, propyl, butyl, pentyl, hexyl, cyclopropyl, cyclobutyl, cyclopentyl, or cyclohexyl is optionally substituted with at least one substituent selected from F, Cl, Br, I, oxo, —CN, —NO₂, methoxy, ethoxy, propoxy, butoxy, pentoxy, hexoxy, heptyloxy, octyloxy, cyclopropyl, cyclobutyl, cyclopentyl, cyclohexyl, cycloheptyl, or cyclooctyl.

Embodiment 3. The compound of embodiment 1, wherein:
R is each independently methyl, ethyl, 1-propyl, or 2-propyl; or
two R together with the two oxygen atoms to which each is attached form a 3-, 5-, 6-, 7-, or 8-membered ring;
R¹ is methyl, ethyl, 1-propyl, or 2-propyl.

Embodiment 4. The compound of embodiment 2, wherein two R together with the two oxygen atoms to which each is attached form a ring which contains 3 carbon atoms or more; and
R¹ is methyl.

Embodiment 5. The compound of embodiment 1, having the structure below:

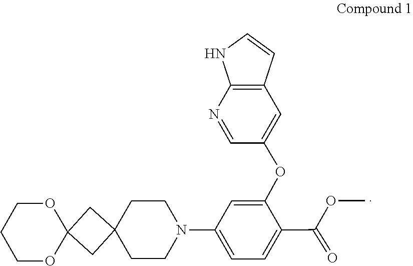 [SMILES: COC(=O)c1ccc(N2CCC3(CC2)CC2(C3)OCCCO2)cc1Oc1cnc2[nH]ccc2c1]

Embodiment 6. A method for preparing the compound of Formula (I) according to any one of embodiments 1-5, said method comprising:
reacting a compound of Formula (II) and a compound of Formula (III) in the presence of base and solvent to form the compound of Formula (I);

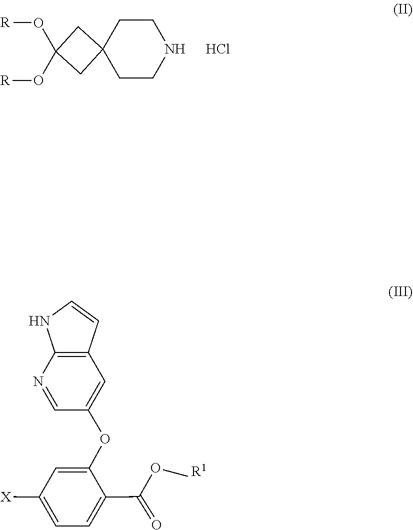

wherein
X is halogen; preferably, X is F, Cl, Br, or I; more preferably, X is F.

Embodiment 7. The method of embodiment 6, wherein the base is selected from the group consisting of: 1,8-Diazabicyclo[5.4.0]undec-7-ene (DBU), 1,1,3,3-Tetramethylguanidine (TMG), 1,5-Diazabicyclo[4.3.0]non-5-ene (DBN), 1,4-Diazabicyclo[2.2.2]octane (DABCO), me-piperidine and N,N-Diisopropylethylamine (DIPEA), or the combination thereof,
preferably, 1,8-Diazabicyclo[5.4.0]undec-7-ene (DBU), 1,1,3,3-Tetramethylguanidine (TMG), 1,5-Diazabicyclo[4.3.0]non-5-ene (DBN), or the combination thereof,
more preferably, 1,8-Diazabicyclo[5.4.0]undec-7-ene (DBU).

Embodiment 8. The method of anyone of embodiments 6-7, wherein the molar ratio of the base used in the reaction to the compound of Formula (II) is 0.9-1.3;
preferably, the molar ratio of the base used in the reaction to the compound of Formula (II) is about 1.

Embodiment 9. The method of anyone of embodiments 6-8, wherein the solvent is selected from the group consisting of: N-methylpyrrolidone (NMP), dimethyl sulfoxide (DMSO), 2-methyltetrahydrofuran (2-MeTHF), 4-methyl-2-pentanone (MIBK) or toluene, or the combination thereof;
preferably, N-methylpyrrolidone(NMP).

Embodiment 10. The method of anyone of embodiments 6-9, wherein the weight ratio of the solvent used in the reaction to the summation of the compound of Formula (II) and the compound of Formula (III) is about 4-6;
preferably, about 5.

Embodiment 11. The method of anyone of embodiments 6-10, wherein the amount of the compound of Formula (II) used in the reaction ranges from about 1.3 molar equivalents to about 1.6 molar equivalents relative to the amount of the compound of Formula (III);
preferably, about 1.45 molar equivalents to about 1.65 molar equivalents.

Embodiment 12. The method of any one of embodiments 6-11, wherein the reaction temperature range is from about 40° C. to about 100° C.;
preferably, about 65° C. to about 80° C.;
more preferably, about 75° C.

Embodiment 13. The method of anyone of embodiments 6-12, said method comprising
Step 1): the compound of Formula (II) and the compound of Formula (III) are reacted in the presence of a base and a solvent;
Step 2): after the reaction of step 1) is completed, water, AcOH and the crystal seed of formula (I) are added to the reaction system; after filtered, the crude product containing the compound of Formula (I) is obtained;
Step 3): the crude product containing the compound of Formula (I) is slurried with a mixture solvent, filtered and dried to obtain the compound of Formula (I).

Embodiment 14. The method of embodiment 13, wherein the mixture solvent comprises 2-MeTHF and n-heptane.

Embodiment 15. The method of embodiment 14, wherein 2-MeTHF: n-heptane(v:v)=1-4:1.

### EXAMPLES

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature to freezing temperature. A given reaction can be carried out in one solvent or a mixture of solvents.

The selection of an appropriate protecting group can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

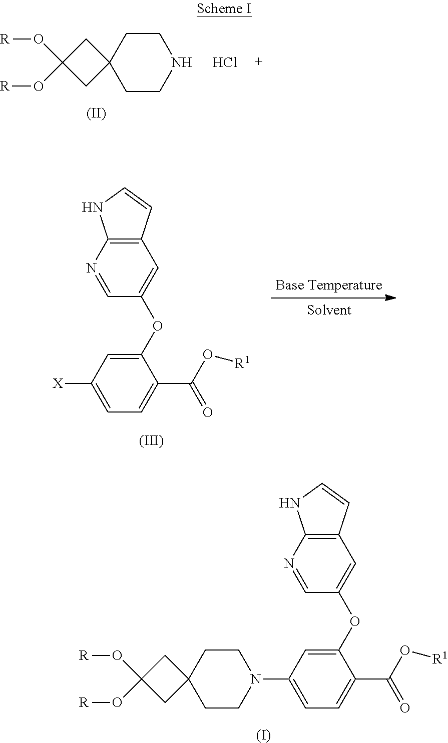

Compounds of Formula (I) can be prepared as shown in Scheme I. Nucleophilic Aromatic Substitution reaction (SNAr) between Compound 2 and Compound 3 with the aid of a base to afford Compound 1. For example:

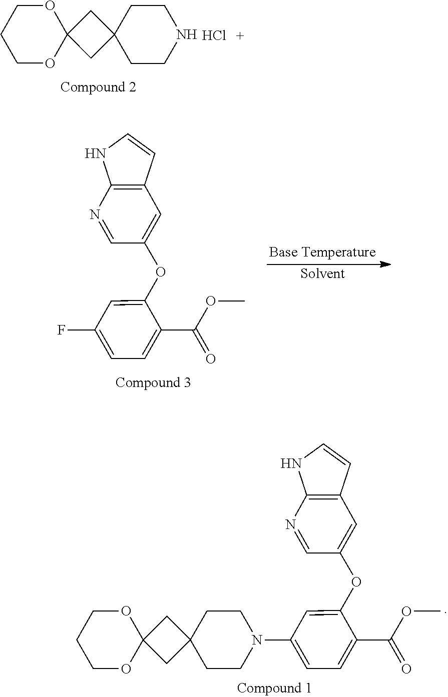 [SMILES: C1COC2(CC3(CCNCC3)C2)OC1 ; Cl ; COC(=O)c1ccc(F)cc1Oc1cnc2[nH]ccc2c1 ; "Solvent" ; COC(=O)c1ccc(N2CCC3(CC2)CC2(C3)OCCCO2)cc1Oc1cnc2[nH]ccc2c1]

Abbreviations

| | |
|---|---|
| SNAr | Nucleophilic Aromatic Substitution reaction |
| AcOH | Acetic Acid |
| DCM | dichloromethane |
| DIPEA | diisopropylethylamine |
| DMAP | 4-dimethylaminopyridine |
| DMF | Dimethylformamide |
| DMSO | Dimethyl sulfoxide |
| EA, EtOAc | Ethyl acetate |
| EDC, EDCI | 1-(3-Dimethylaminopropyl)-3-ethylcarbodiimide |
| | hydrochloride; N-Ethyl-N′-(3- |
| | dimethylaminopropyl)carbodiimide hydrochloride |
| Eq. | equivalent |
| Et₃N, TEA | triethyl amine |
| EtOH | ethanol |
| Fe | iron |
| HATU | 1-[bis(dimethylamino)methylene]-1H-1,2,3-triazolo[4,5- |
| | b]pyridinium 3-oxid hexafluorophosphate |
| HCl | Hydrochloric Acid |
| HOBT | 1-Hydroxybenzotriazole |
| MeCN | acetonitrile |
| MeOH | Methanol |
| MIBK | 4-methyl-2-pentanone |
| 2-MeTHF | 2-methyltetrahydrofuran |
| NaBH₄ | Sodium borohydride |
| NBS | N-Bromosuccinimide |
| NMP | N-methylpyrrolidone |
| Pd(dppf)Cl₂ | [1,1′-Bis(diphenylphosphino)ferrocene]dichloropal- |
| | ladium(II) |
| Pd(OAc)₂ | Palladium(II)acetate |
| PE | petroleum ether |
| PreP-HPLC | Preparative High-Performance Liquid Chromatography |
| Prep-TLC | Preparative thin-layer chromatography |
| rt | room temperature |
| t-BuONO | tert-Butyl nitrite |
| TFA | trifluoroacetic acid |
| TLC | thin-layer chromatography |
| IPAc | Isopropyl acetate |
| TEOF | Triethyl orthoformate |
| MTBE | Methyl Tertiary Butyl Ether |
| LDPE | Low Density Polyethylene |
| NMR | Nuclear Magnetic Resonance Spectroscopy |
| UV | Ultraviolet |
| HPLC | High Performance Liquid Chromatography |
| LC-MS | Liquid Chromatograph Mass Spectrometer |
| GC | Gas chromatography |

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

HPLC: Waters with UV detector or equivalent. Mobile phase: A: water with 0.01% TFA, B: acetonitrile with 0.01% TFA. Column: Waters CORTECS C18+, 100*4.6 mm, 2.7 um, PN: 186007407. Gradient method: Flow: 1.0 mL/min Time (min) A (%) B (%)

| Time (min) | A(%) | B(%) |
|---|---|---|
| 0 | 80 | 20 |
| 19 | 55 | 45 |
| 31 | 10 | 90 |
| 31.1 | 80 | 20 |
| 36 | 80 | 20 |

### Control Example:

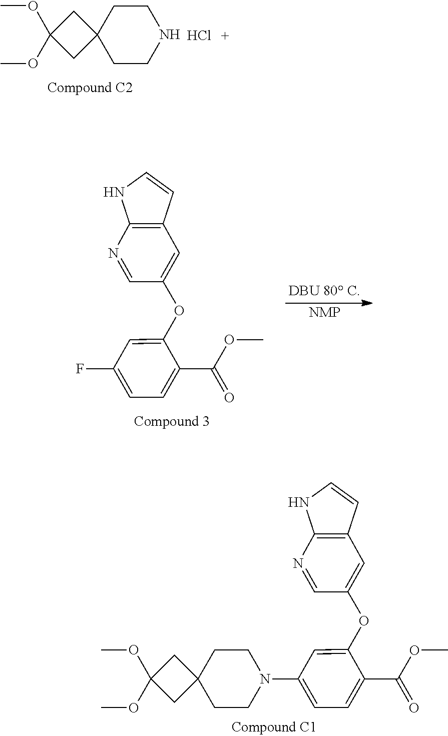

Compound C1 was prepared by the reaction between Compound C2 and Compound 3. However, Compound C1 was found not stable, and it was easy to be deprotected. Therefore, it cannot be a useful intermediate to prepare Sonrotoclax.

Compound C1 was prepared as below.

To a suspension of methyl 2-((1H-pyrrolo[2,3-b]pyridin-5-yl)oxy)-4-fluorobenzoate (1.01 g, 3.49 mmol) and 2,2-dimethoxy-7-azaspiro[3.5]nonane hydrochloride (1.06 g, 4.54 mmol, prepared by the method disclosed in WO2019/210828) in NMP (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. MS: 451.2.

### Example A1: 1,5-dioxa-11-azadispiro[5.1.5.1]tetradecane hydrogen chloride

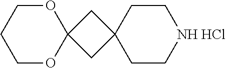

Tert-butyl 2-oxo-7-azaspiro[3.5]nonane-7-carboxylate (10.0 g, 41.8 mmol) was charged in a nitrogen inerted reactor. To the reactor was added IPAc (20.0 ml) to obtain a mixture. The mixture was stirred at 20-30° C. for 1 h. To this mixture was added triethyl orthoformate (8.1 g, 54.7 mmol) and then 1,3-propylene glycol (10.0 g, 129.6 mmol). The reactor was fed with 15% HCl/IPAc solution (36.0 g, 146.3 mmol), while keeping the batch temperature at 20-30° C. for approximately 20 hrs. The product was tested by GC (carrier gas: helium (He)) which shows that 99.9% of 1,5-dioxa-11-azadispiro[5.1.5.1]tetradecane hydrogen chloride was obtained and the solid was well separated out. GC-MS: 197.1

### Example A2: 1,5-dioxa-11-azadispiro[5.1.5.1]tetradecane hydrogen chloride

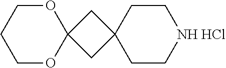

Tert-butyl 2-oxo-7-azaspiro[3.5]nonane-7-carboxylate (10.0 g, 41.8 mmol) was charged in a nitrogen inerted reactor. To the reactor was added IPAc (20.0 ml) to obtain a mixture. The mixture was stirred at 20-30° C. for 1 h. To this mixture was added triethyl orthoformate (8.1 g, 54.7 mmol) and then 1,3-propylene glycol (10.0 g, 129.6 mmol). The reactor was fed with of 15% HCl/IPAc solution (36.0 g, 146.3 mmol) while keeping the batch temperature at 20-30° C. for approximately 16 hours. Upon reaction completion, the batch is charged with Methyl Tertiary Butyl Ether (MTBE, 27 ml) while keeping the batch temperature at 25±5° C. The mixture is aged for approximately 6 hrs and subsequently centrifuged to collect the wet cake. The cake is washed with MTBE (27 ml). The wet cake is dried under vacuum at 40±5° C. for 20 h to obtain solid product 1,5-dioxa-11-azadispiro[5.1.5.1]tetradecane hydrogen chloride. ¹H NMR (400 MHz, d-DMSO) δ 8.908 (s, 2H), 3.722 (t, 4H), 2.924 (m, 4H), 1.906 (s, 4H), 1.692 (t, 4H), 1.554 (t, 2H). GC-MS: 197.1

### Example A3: Stability Test of 1,5-dioxa-11-azadispiro[5.1.5.1]tetradecane hydrogen chloride

1,5-dioxa-11-azadispiro[5.1.5.1]tetradecane hydrogen chloride (2.0 g, 8.4 mmol) was stored in 2-8° C. and the packaged in double layered LDPE bag with desiccant between the two layers, inside a heat-sealed aluminum foil bag for 3.5 months. The sample was tested by GC (carrier gas: N2) and HPLC (Column: Waters Xselect HSS T3(150 mm*4.6 mm,3.5 um) P/N:186004786; mobile phase 0.05% TFA in water, ACN:MeOH=1:1, v/v; Flow: 1 ml/min; Temperature: 30° C.). The result in Table 1 shows that intermediate 1,5-dioxa-11-azadispiro[5.1.5.1]tetradecane hydrogen chloride was stable enough in this condition.

TABLE 1

Product, Impurity profile (RRT, Area %)

| | | | |
|---|---|---|---|
| Storage condition | 1.00 | | |
| 2~8° C. for 3.5 | 99.90 | / | / |
| Months | | | |

### Example 1: NMP as Solvent, DBU as Base, Reaction Temperature: 80° C. And 1.3 Equivalent of Compound 2, which was Relative to the Equivalent of Compound 3

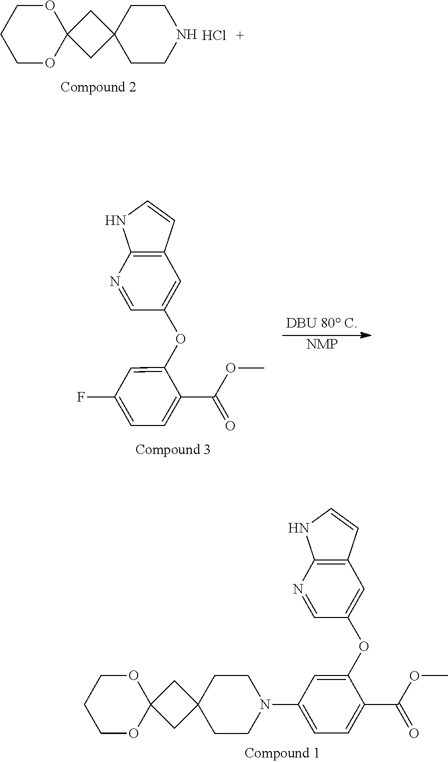

Methyl 2-((1H-pyrrolo[2,3-b]pyridin-5-yl)oxy)-4-fluorobenzoate was synthesized per the methods in WO2019210828 (e.g., Example F43), which is incorporated by reference in its entirety.

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.06 g, 4.54 mmol) in NMP (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 93.17%, yield: 83%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 2: NMP as Solvent, DBU as Base, Reaction Temperature: 80° C. And 1.45 Equivalent of Compound 2, which was Relative to the Equivalent of Compound 3

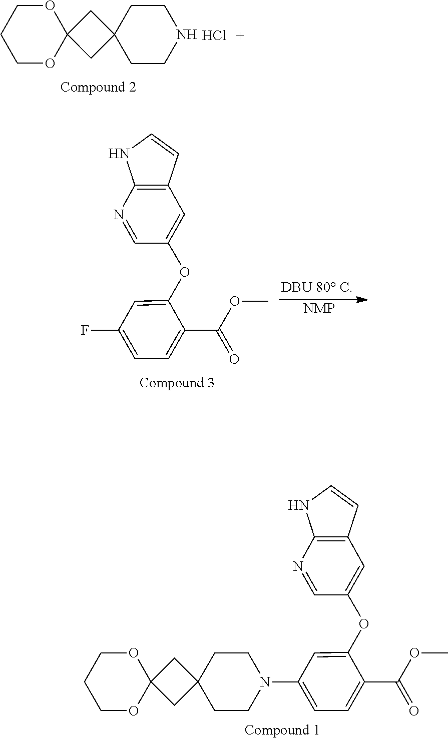

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.18 g, 5.06 mmol) in NMP (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 90.59%, yield: 87%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 3: NMP as Solvent, DBU as Base, Reaction Temperature: 80° C. And 1.60 Equivalent of Compound 2, which was Relative to the Equivalent of Compound 3

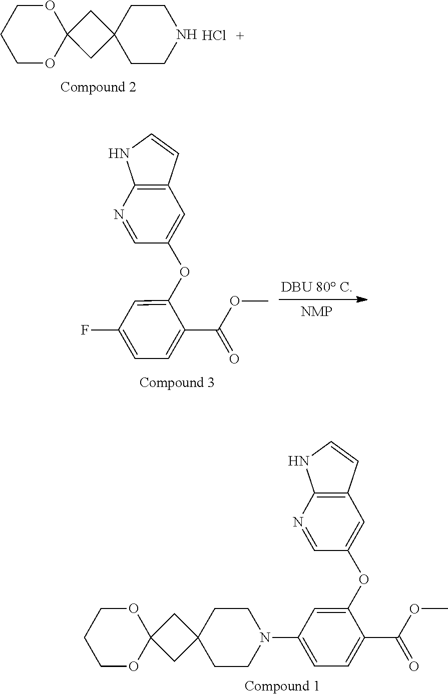 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 93.91%, yield: 86%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 4: NMP as Solvent, DBU as Base, Reaction Temperature: 60° C. And 1.60 Equivalent of Compound (I), which was Relative to the Equivalent of Compound (II)

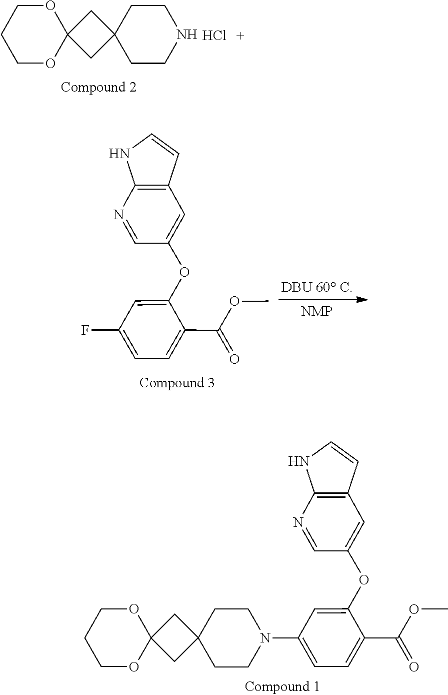 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 60° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 93.83%, yield: 82%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 5: NMP as Solvent, DBU as Base, Reaction Temperature: 65° C. And 1.60 Equivalent of Compound 2, which was Relative to the Equivalent of Compound 3

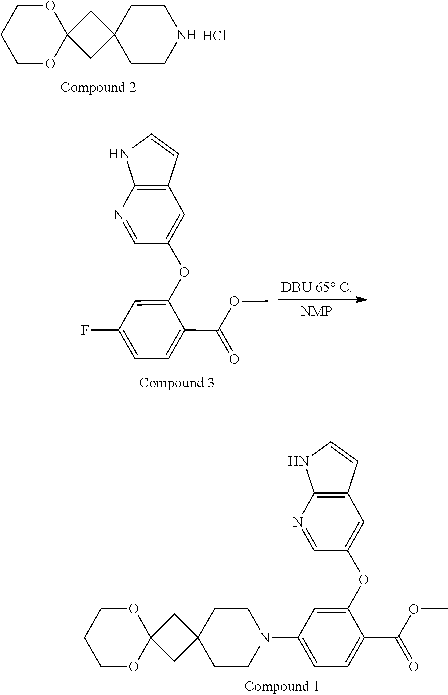 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 65° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.62%, yield: 83%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 6: NMP as Solvent, DBU as Base, Reaction Temperature: 70° C. And 1.60 Equivalent of Compound (I), which was Relative to the Equivalent of Compound (II)

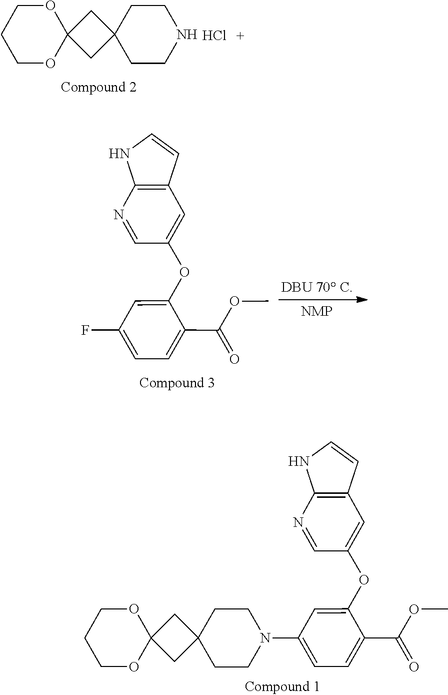 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 70° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.26%, yield: 83%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 7: NMP as Solvent, DBU as Base, Reaction Temperature: 75° C. And 1.60 Equivalent of Compound 2, which was Relative to the Equivalent of Compound 3

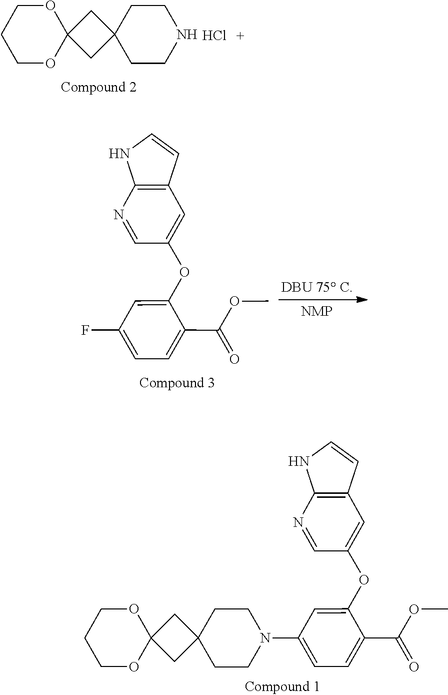 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 75° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.25%, yield: 81%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 8: NMP as Solvent, DBU as Base, Reaction Temperature: 80° C. And 1.60 Equivalent of Compound 2, which was Relative to the Equivalent of Compound 3

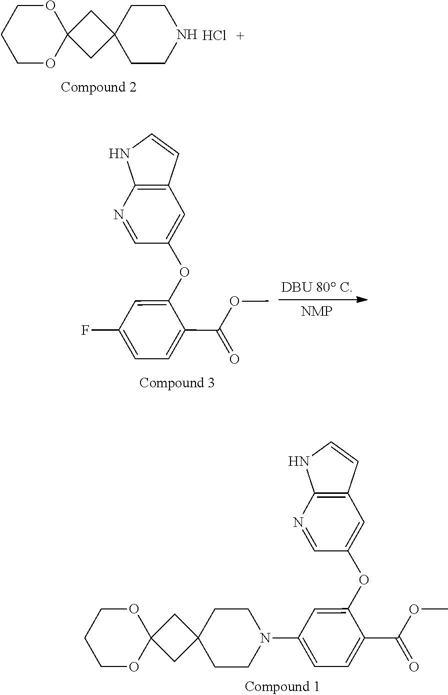 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 94.27%, yield: 83%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 9: DMSO as Solvent, DBU as Base, Reaction Temperature: 80° C. And 1.60 Equivalent of Compound 2, which was Relative to the Equivalent of Compound 3

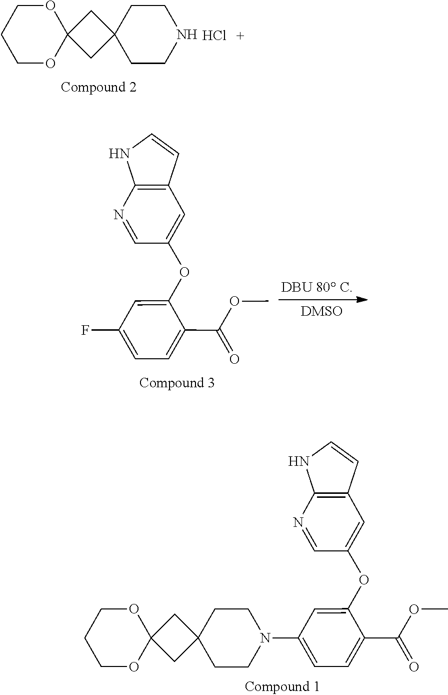 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in DMSO (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 94.27%, yield: 81%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 10: 2-MeTHF as Solvent, DBU as Base, Reaction Temperature: 80° C. And 1.60 Equivalent of Compound 2, which was Relative to the Equivalent of Compound 3

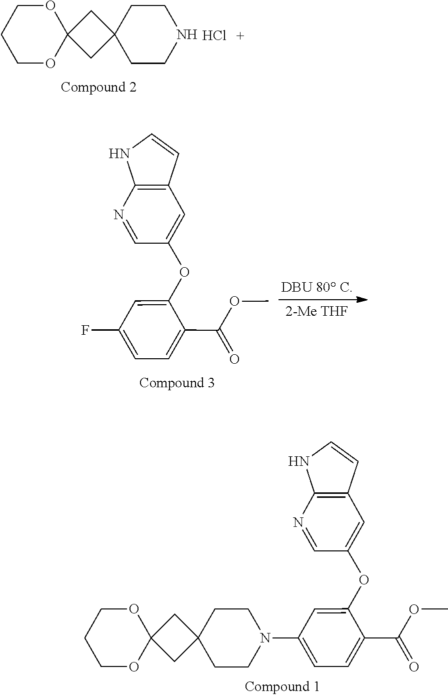 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in 2-MeTHF (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 80.18%, yield: 71%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 11: MIBK as Solvent, DBU as Base, Reaction Temperature: 80° C. And 1.60 Equivalent of Compound 2, which was Relative to the Equivalent of Compound 3

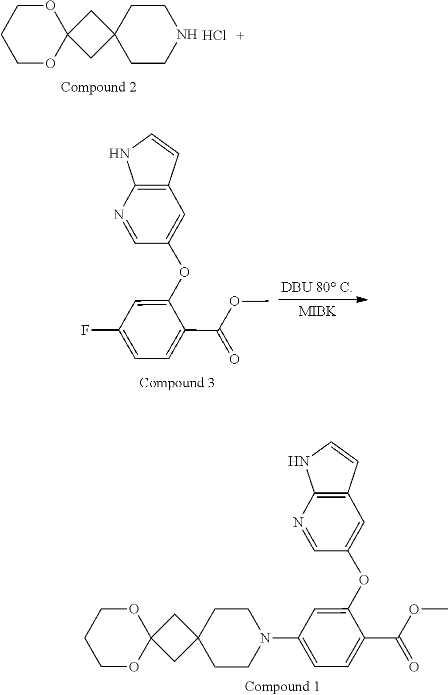 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in MIBK (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 90.91%, yield: 78%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 12: Toluene as Solvent, DBU as Base, Reaction Temperature: 80° C. And 1.60 Equivalent of Compound 2, which was Relative to the Equivalent of Compound 3

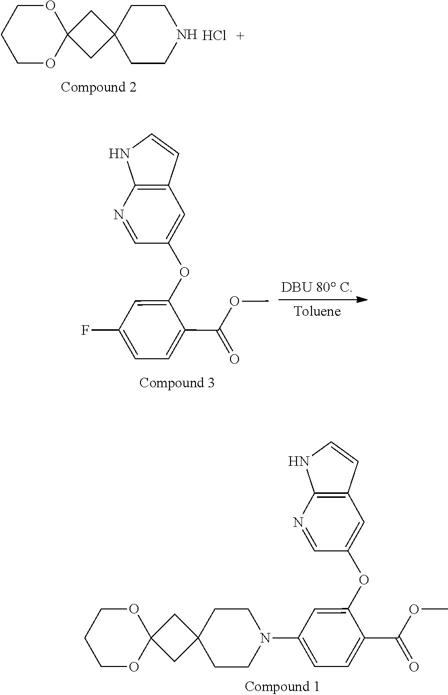 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in Toluene (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 87.31%, yield: 76%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 13: DMAc as Solvent, DBU as Base, Reaction Temperature: 80° C. And 1.60 Equivalent of Compound 2, which was Relative to the Equivalent of Compound 3

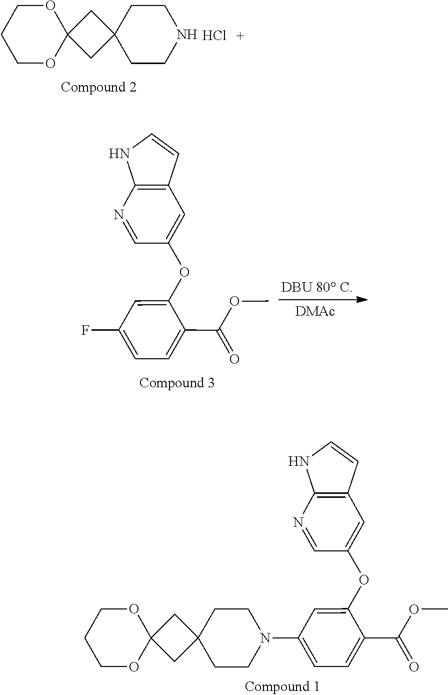

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in DMAc (5 mL), followed by the addition of DBU (1.50 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 94.36%, yield: 83%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 14: NMP as Solvent, TMG as Base, Reaction Temperature: 80° C. And 1.60 Equivalent of Compound 2, which was Relative to the Equivalent of Compound 3

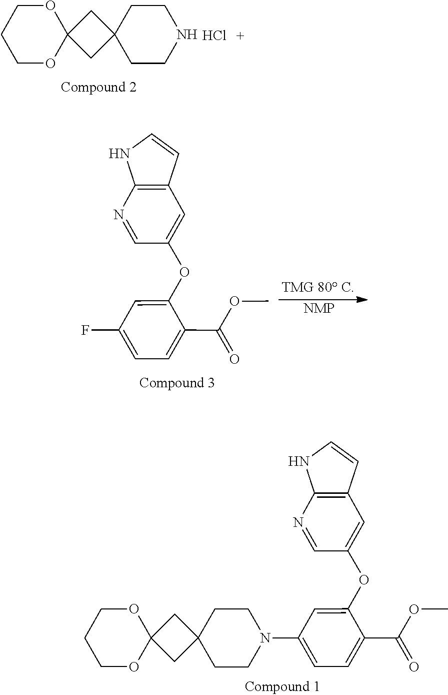

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of TMG (1.13 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 87.51%, yield: 80%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 15: NMP as Solvent, DBN as Base, Reaction Temperature: 80° C. And 1.60 Equivalent of Compound 2, which was Relative to the Equivalent of Compound 3

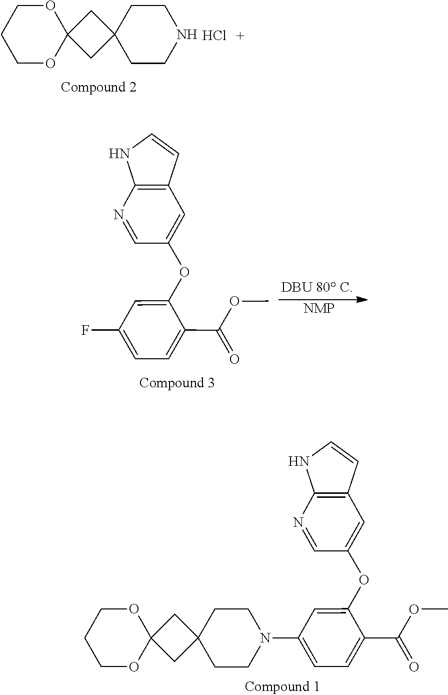

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBN (1.22 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 5.5%, yield: N.D. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 16: NMP as Solvent, DABCO as Base, Reaction Temperature: 80° C. And 1.60 Equivalent of Compound 2, which was Relative to the Equivalent of Compound 3

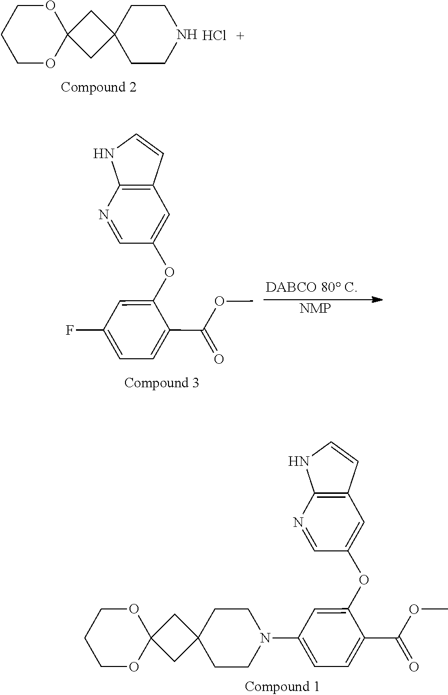

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DABCO (1.10 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 8.9%, yield: 5%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 17: NMP as Solvent, Me-Piperidine as Base, Reaction Temperature: 80° C. And 1.60 Equivalent of Compound 2, which was Relative to the Equivalent of Compound 3

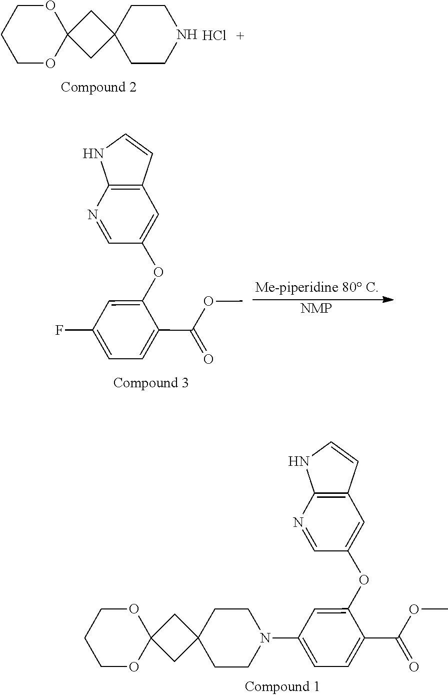

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of Me-piperidine (0.98 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 84.38%, yield: 70%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 18: NMP as Solvent, DIPEA as Base, Reaction Temperature: 80° C. And 1.60 Equivalent of Compound 2, which was Relative to the Equivalent of Compound 3

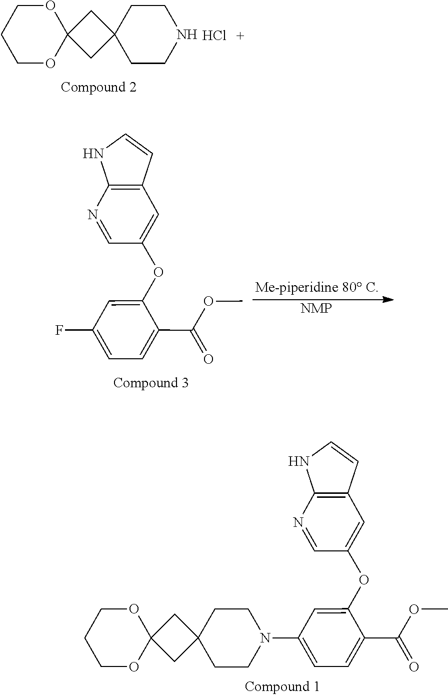

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DIPEA (1.27 g, 9.85 mmol). The resulting solution was stirred for 24 h at 80° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 86.18%, yield: 76%. 1H NMR (400 MHz, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 19: 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Sonrotoclax)

Step 1: methyl 2-((1H-pyrrolo[2,3-b]pyridin-5-yl)oxy)-4-(2-oxo-7-azaspiro[3.5]nonan-7-yl)benzoate

To the solution of methyl 2-((1H-pyrrolo[2,3-b]pyridin-5-yl)oxy)-4-(1,5-dioxa-11-azadispiro[5.1.5⁸.1⁶]tetradecan-11-yl)benzoate (180 g, 0.39 mol) in DCM (2 L) was added diluted HCl acid (1M, 1.5 L) and stirred for overnight. After the reaction was completed, the mixture was cooled to 10° C. and was adjusted to pH=8-9 with aqueous NaOH solution (4 M) under stirring. The organic phase was separated and washed with 15% aq. NaCl (1 L), then washed with H₂O (1 L). After the organic phase was concentrated to 500 mL, MTBE (1 L) was poured into the solution and then the system was concentrated to 500 mL (repeated this work-up 3 times). The resulting system was stirred for 0.5 hour. After filtration, the cake was collected and then dried in vacuum to obtain the tittle product as a white solid (152 g, yield: 96%). ¹H NMR (400 MHz, DMSO-d₆) δ ppm: 11.64 (s, 1H), 8.02 (d, J=2.4 Hz, 1H), 7.78 (d, J=9.2 Hz, 1H), 7.47 (t, J=3.2 Hz, 1H), 7.44 (d, J=2.4 Hz, 1H), 6.83 (dd, J=2.4 Hz, J=9.2 Hz, 1H), 6.43 (d, J=2.4 Hz, 1H), 6.38-6.36 (m, 1H), 3.65 (s, 3H), 3.24-3.21 (m, 4H), 2.80 (s, 4H), 1.70-1.67 (m, 4H). MS (ESI, m/e) [M+1]⁺ 405.9.

Step 2: (S)-tert-butyl 2-(2-(prop-1-en-2-yl)phenyl)pyrrolidine-1-carboxylate

To a mixture of (S)-tert-butyl 2-(2-bromophenyl)pyrrolidine-1-carboxylate (50 g, 153.3 mmol) and 4,4,5,5-tetramethyl-2-(prop-1-en-2-yl)-1,3,2-dioxaborolane (38.6 g, 229.9 mmol) in dioxane (500 mL) and H₂O (50 mL) was added Cs₂CO₃ (100 g, 305 mmol) and Pd(dppf)Cl₂ (6.6 g, 7.5 mmol). The mixture was stirred at 100° C. for 8 hours. TLC showed the reaction was completed. The mixture was concentrated in vacuum. The residue was purified by column chromatography on silica gel (eluent: PE/EA (v/v)=100/1 to 10/1) to obtain (S)-tert-butyl 2-(2-(prop-1-en-2-yl)phenyl)pyrrolidine-1-carboxylate (65 g, crude). The crude product was used directly in next step.

Step 3: (S)-tert-butyl 2-(2-isopropylphenyl)pyrrolidine-1-carboxylate

To a solution of (S)-tert-butyl 2-(2-(prop-1-en-2-yl)phenyl)pyrrolidine-1-carboxylate (30 g, 104.39 mmol) in MeOH (500 mL) was added Pd/C (10 g, 10%) and the mixture was stirred at 20° C. under H₂ (15 Psi) for 12 hours. TLC showed the reaction was completed. The mixture was filtered and the filtrate was concentrated in vacuum to give (S)-tert-butyl 2-(2-isopropylphenyl)pyrrolidine-1-carboxylate (60 g, crude), which was used in next step without further purification. ¹H NMR (400 MHz, CDCl₃) δ ppm: 7.39-6.90 (m, 4H), 5.36-5.04 (m, 1H), 3.77-3.52 (m, 2H), 3.20-3.17 (m, 1H), 2.47-2.24 (m, 1H), 1.96-1.65 (m, 3H), 1.54-1.38 (m, 2H), 1.31-1.22 (m, 8H), 1.17 (s, 7H).

Step 4: (S)-2-(2-isopropylphenyl)pyrrolidine hydrochloride

To a solution of tert-butyl 2-(2-isopropylphenyl)pyrrolidine-1-carboxylate (55 g, 190 mmol) in DCM (50 mL) was added HCl in 1,4-dioxane (4 M, 142 mL, 570 mmol) dropwise at room temperature. The mixture was stirred at room temperature for overnight. The mixture was concentrated in vacuum. The resulting residue was slurried with EA (100 mL) and then filtered, dried in vacuum to give (S)-2-(2-isopropylphenyl)pyrrolidine hydrochloride 26 g (yield: 60.4%). ¹H NMR (400 MHz, DMSO-d₆) δ ppm: 9.93 (s, 1H), 8.81 (s, 1H), 7.63-7.57 (m, 1H), 7.41-7.34 (m, 2H), 7.32-7.24 (m, 1H), 4.91-4.75 (m, 1H), 3.47-3.35 (m, 1H), 3.31-3.25 (m, 1H), 2.40-2.21 (m, 1H), 2.19-1.86 (m, 3H), 1.25 (d, J=6.7 Hz, 3H), 1.17 (d, J=6.7 Hz, 3H). MS (ESI, m/e) [M+1]+190.0.

Step 5: methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate

A mixture of (S)-2-(2-isopropylphenyl)pyrrolidine hydrochloride (120 g, 0.535 mole) and methyl 2-((1H-pyrrolo[2,3-b]pyridin-5-yl)oxy)-4-(2-oxo-7-azaspiro[3.5]nonan-7-yl)benzoate (218 g, 0.509 mole) in DCM (2.2 L) was charged into a reactor. The temperature was controlled below 30° C. and NaBH(OAc)₃ (216 g, 1.018 mole) was added into the reactor in 5-6 portions. Then the reaction mixture was stirred at room temperature and monitored by TLC. After the starting material ketone was consumed completely, the mixture was adjusted to pH=4-5 with diluted HCl acid (0.5 M). The separated organic phase was washed with H₂O (600 mL×2) and then washed with aq. NaHCO₃ (600 mL×2), saturated aq. NaCl (600 mL). The organic phase was collected, then dried over anhydrous Na₂SO₄ and concentrated. 256 g off-white solid was obtained as the crude product, which was used in the next step directly. MS (ESI, m/e) [M+1]⁺ 579.0.

Step 6: (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid

To a solution of methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate (105 g, 181.7 mmol) in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5-6 with 3 N HCl acid at room temperature with stirring. The precipitate was filtered and dried in vacuum to give the product as a white solid (102.4 g, yield: 99%). ¹H NMR (400 MHz, DMSO-d₆) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1H), 2.9-2.15 (m, 1H), 1.74-1.64 (m, 4H), 1.52-1.37 (m, 6H), 1.28-1.06 (m, 6H). MS (ESI, m/e) [M+1]+564.9.

Step 7: 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Sonrotoclax)

A mixture of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol) in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid was consumed completely, the reaction mixture was heated to ˜35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to room temperature, the precipitate was filtered and then the wet cake was washed twice with EA (180 mL). After drying in vacuum at 80-90° C., the desired compound was obtained (48 g, yield: 69.5%). ¹H NMR (DMSO-d₆) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]+889.9.

What is claimed is:

1. A compound having the structure below: 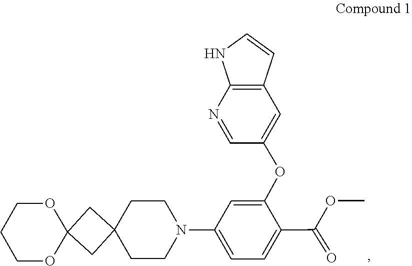 [SMILES: COC(=O)c1ccc(N2CCC3(CC2)CC2(C3)OCCCO2)cc1Oc1cnc2[nH]ccc2c1]
or a salt thereof.

2. A method of providing a compound of Formula (SI), 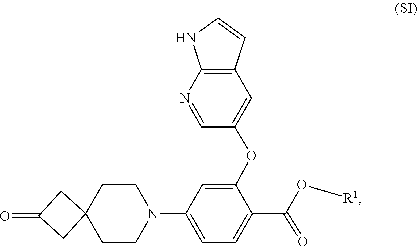
or a salt thereof, comprising reacting a compound of Formula (I) 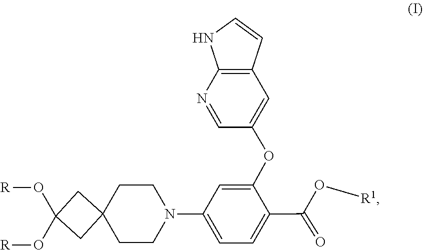
or a salt thereof, with an acid or base, wherein,
at each of occurrences, R is each independently C₂₋₆alkyl or C₃₋₆cycloalkyl, each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
R¹ is C₁₋₆alkyl, or C₃₋₆cycloalkyl, wherein each of said C₁₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl.

3. The method of claim 2, wherein the acid is HCl, or the base is NaOH.

4. The method of claim 2 or 3, further comprising reacting the compound of Formula (SI) with (S)-2-(2-isopropylphenyl)pyrrolidine, or a salt thereof, to provide a compound of Formula (SII) 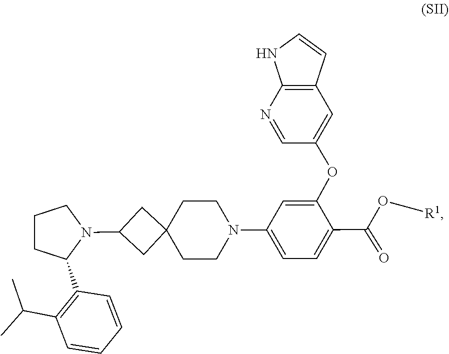
or a salt thereof.

5. The method of claim 4, comprising reacting the compound of Formula (SI) with (S)-2-(2-isopropylphenyl)pyrrolidine, or a salt thereof, in the presence of NaBH(OAc)₃.

6. The method of claim 4 or 5, comprising reacting the compound of Formula (SI) with (S)-2-(2-isopropylphenyl)pyrrolidine, or a salt thereof, at a temperature less than about 30° C.

7. The method of any one of claims 4-6, further comprising reacting the compound of Formula (SII) 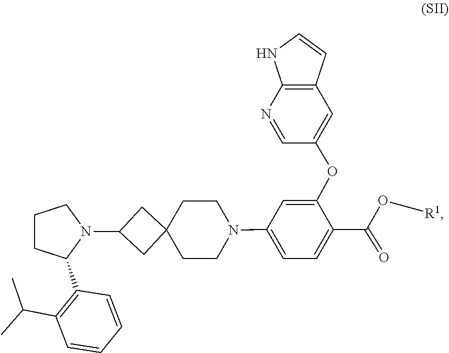
or a salt thereof, with 4-((((1r,4r)-4-hydroxy-4-methylcyclohexyl)methyl)amino)-3-nitrobenzenesulfonamide, to provide Sonrotoclax, 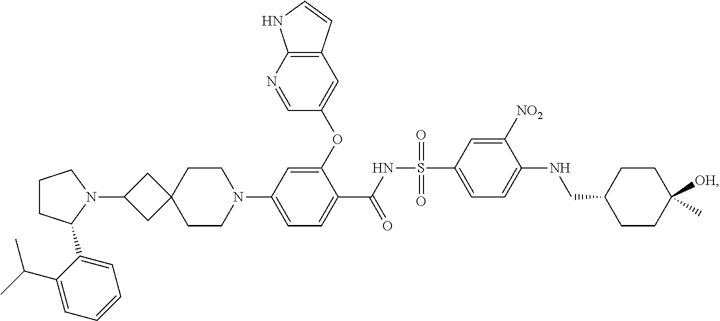 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof.

8. The method of claim 7, comprising reacting the compound of Formula (SII) or a salt thereof, with 4-((((1r,4r)-4-hydroxy-4-methylcyclohexyl)methyl)amino)-3-nitrobenzenesulfonamide, in the presence of EDCI and DMAP.

9. The method of any one of claims 4-64, further comprising reacting the compound of Formula (SII), or a salt thereof, with an acid or a base, to provide (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, 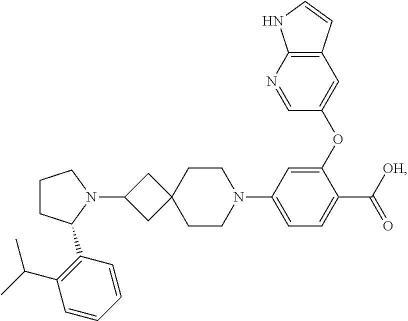 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof.

10. The method of claim 9, wherein the acid is HCl, or the base is NaOH.

11. The method of claim 9 or 10, further comprising reacting (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, 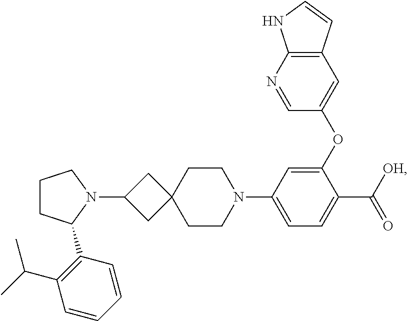 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof, with 4-((((1r,4r)-4-hydroxy-4-methylcyclohexyl)methyl)amino)-3-nitrobenzenesulfonamide, to provide Sonrotoclax, 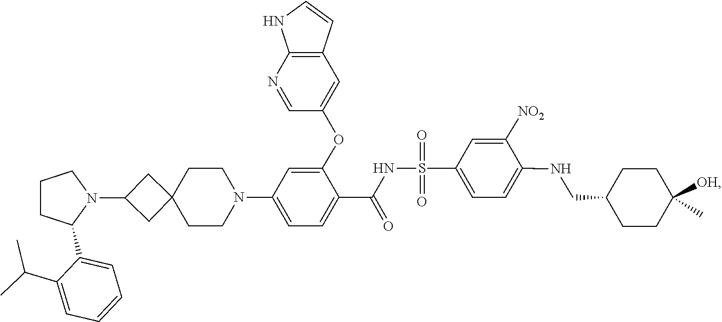 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof.

12. The method of claim 11, comprising reacting (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, or a salt thereof, with 4-((((1r,4r)-4-hydroxy-4-methylcyclohexyl)methyl)amino)-3-nitrobenzenesulfonamide, in the presence of EDCI and DMAP.

13. A method for preparing a pharmaceutical composition comprising Sonrotoclax, comprising mixing Sonrotoclax with a pharmaceutically acceptable excipient, wherein Sonrotoclax is prepared according to the method of claim 7 or 8.

14. A method for preparing a pharmaceutical composition comprising Sonrotoclax, comprising mixing Sonrotoclax with a pharmaceutically acceptable excipient, wherein Sonrotoclax is prepared according to the method of any one of claims 9-11.

15. A pharmaceutical composition comprising sonrotoclax or a pharmaceutically acceptable salt thereof and a pharmaceutically acceptable excipient, wherein sonrotoclax is prepared according to the method of any one of claims 2-12.
